(19) United States
(12) Patent Application Publication
(10) Pub. No.: US 2021/0267554 A1
(43) Pub. Date: Sep. 2, 2021

(54) ESTABLISHMENT OF A BASELINE MEASUREMENT OF RESPIRATORY ACTIVITY FOR EFFICIENT MEASUREMENT OF CHANGES

(71) Applicant: MAKANI SCIENCE INC., Irvine, CA (US)

(72) Inventors: Francis Duhay, Irvine, CA (US); Michael Chu, Irvine, CA (US); William E. Saltzstein, Irvine, CA (US)

(21) Appl. No.: 17/184,982

(22) Filed: Feb. 25, 2021

Related U.S. Application Data
(60) Provisional application No. 62/984,109, filed on Mar. 2, 2020.

(51) Int. Cl. A61B 5/00; G08B 21/18; G08B 21/04; A61B 5/113; A61B 5/08; G16H 40/67; G16H 50/30; G16H 10/60

(52) U.S. Cl. CPC A61B 5/7282; G08B 21/182; G08B 21/0453; A61B 5/1135; A61B 5/0803; A61B 5/746; A61B 5/091; A61B 5/0024; G16H 40/67; G16H 50/30; G16H 10/60; A61B 2562/0261; A61B 2562/0219; A61B 5/6823

(57) ABSTRACT

The present invention is directed to the measurement of a baseline respiratory level in a patient so that changes in respiratory activity can be easily reported. The baseline measurement is collected by a sensor in the patient's chest and a sensor on the patient's abdomen, and is transmitted to a computing device. The computing device measures normal breathing for 60 seconds and uses the waveform collected over this period of time to generate a baseline RR, tidal volume, and minute ventilation. From this point, the patient's RR, tidal volume, and minute ventilation are recorded and compared to the baseline measurements, and if a change from the baseline measurement that exceeds a predetermined threshold is detected, the doctor is alerted that action must be taken for the patient's safety.

### CROSS-REFERENCES TO RELATED APPLICATIONS

This application is a non-provisional and claims benefit of U.S. Provisional Application No. 62/984,109 filed Mar. 2, 2020, the specification of which is incorporated herein in its entirety by reference.

### BACKGROUND OF THE INVENTION

The measurement of respiratory data in a patient can be used to track the patient's breathing habits, detect the presence of a lung related ailment, and alert a doctor if a breathing issue arises while the patient is under anesthesia. For these reasons, accurate and efficient methods for measuring respiratory activity in real-time is a priority in the medical world. Prior methods require doctors to measure the absolute tidal volume (ATV) of a patient's lungs using a secondary device (spirometer) for calibration, and many factors such as body habitus, age, and sex must be taken into account when determining the threshold for potentially hazardous levels for patient safety. This method is time-consuming and leaves a significant amount of room for error. Thus, a present need exists for accurate and time-efficient measurement of a patient's respiratory activity and determination of the threshold of hazardous respiratory levels without the need for calibration or the use of a secondary device.

### FIELD OF THE INVENTION

The present invention is directed to the measurement of a “baseline” respiratory level in a patient and the subsequent measurement of “relative” respiratory levels in respect to said baseline level in order to alert a doctor if said patient reaches a hazardous level of respiratory activity above or below the baseline measurement.

### BRIEF SUMMARY OF THE INVENTION

It is an objective of the present invention to provide a method that allows for the measurement of a baseline respiratory level in a patient and the subsequent measurement of relative respiratory levels based on said baseline value, as specified in the independent claims. Embodiments of the invention are given in the dependent claims. Embodiments of the present invention can be freely combined with each other if they are not mutually exclusive.

The method may comprise attaching two respiratory measurement sensors to the skin of a patient's chest and abdomen. The respiratory measurement sensors may be communicatively coupled to a computing device, said computing device being capable of generating and analyzing a waveform based on the data received from said sensors. A patient's normal breathing activity may be measured to generate a baseline measurement of the patient's respiratory levels to which all subsequent respiratory events will be measured against. The doctor may then choose the dominant breathing type (chest or abdomen) to use to generate the baseline measurement. The baseline measurement may comprise a respiratory rate (RR), a baseline tidal volume (BTV), and a baseline minute ventilation (BMV). A hazard threshold may set as a fixed percentage above or below the baseline measurement, and all subsequent respiratory measurements may be compared to the baseline method to determine whether or not the hazard threshold is passed. If the hazard threshold is passed, the doctor may be alerted by the computing device that intervention is required for the patient's safety. Without wishing to limit the present invention to any theory or mechanism, it is believed that the technical feature of the present invention advantageously provides for an accurate, simple, and time-efficient method of measuring a patient's changes in respiratory activity without the need for calibration or the use of a secondary device. This is because the prior need to calibrate the sensors or computing device to a spirometer and the need to factor in body habitus, age, sex, etc in a patient is replaced with the simpler method of generating a baseline measurement for every individual patient. Furthermore, the present invention focuses on measuring the patient's changes in respiratory activity relative to their normal respiratory activity which is more helpful to doctors and more time-efficient to report than measurements of raw data as seen in prior works.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows an embodiment of the method of the present invention, wherein data is read from respiratory measurement sensors to an iOS platform application through Bluetooth communication, a dominant breathing type is selected from the application, and measured changes from a generated baseline measurement are wirelessly transmitted or are printed.

### DETAILED DESCRIPTION OF THE INVENTION

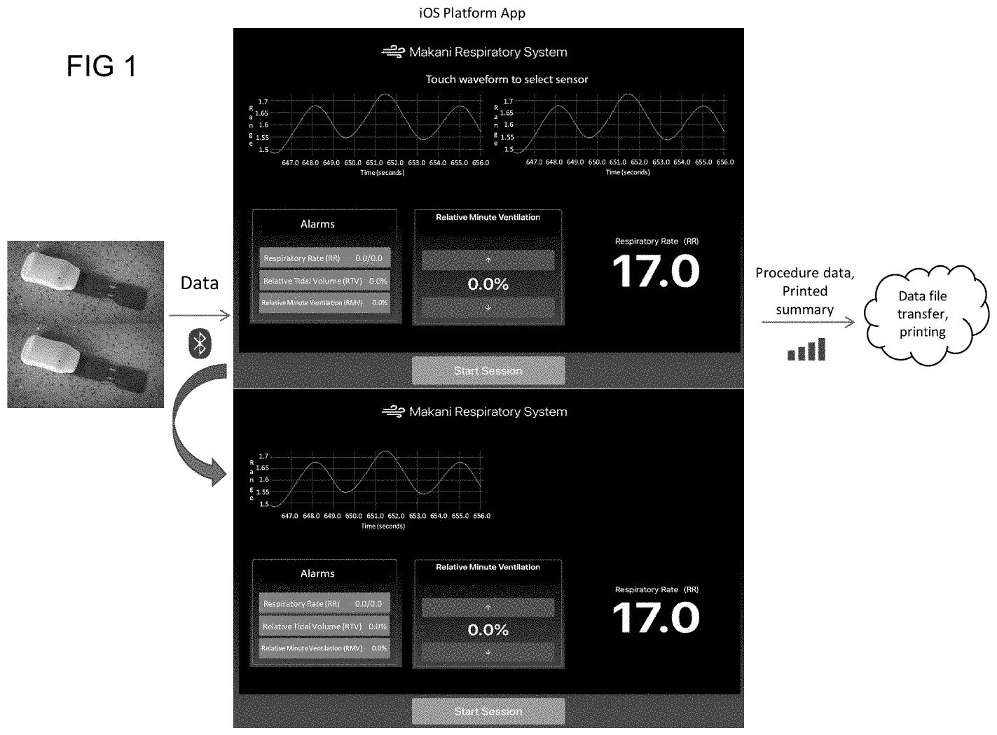

Referring now to FIG. 1, the present invention features a method for measuring a baseline respiratory level in a patient and measuring subsequent changes from said baseline methods in order to alert a doctor of potentially hazardous respiratory levels. The method may comprise applying a first respiratory measurement sensor to the skin of the patient's chest, and applying a second respiratory measurement sensor to the skin of the patient's abdomen. In some embodiments, the first respiratory measurement sensor and the second respiratory measurement sensor may be attached to the skin by an adhesive layer and may be communicatively coupled to a computing device through a wired connection or a wireless connection. The computing device may be a software application on a computer or a software application on a mobile device. In some embodiments, the computing device may be selected from a group comprising the plurality of strain sensors, an external computing device, a cloud server, and a combination thereof. In some embodiments, the first and second respiratory measurement sensors comprise a first and a second strain sensor capable of measuring a physical signal from a respective location on the body. Each physical signal may represent an expansion and contraction measurement of the respective location. In some embodiments, the first and the second strain sensors may be capable of measuring a processed raw physical signal from a respective location on the body. In some embodiments, the method comprises applying a plurality of strain sensors to a plurality of locations on the patient's body for transmitting a plurality of physical signals to the computing device. The plurality of strain sensors may comprise a first strain sensor attached to a chest of the patient, and a second strain sensor attached to an abdomen of the patient.

In some embodiments, the method may further comprise the plurality of strain sensors measuring the plurality of physical signals. Each physical signal may comprise 30 to 60 seconds of respiratory activity at the respective location that the corresponding strain sensor is attached to. In some embodiments, each physical signal may comprise 5 to 60 seconds of respiratory activity at the respective location that the corresponding strain sensor is attached to. In some embodiments, each physical signal may comprise less than 60 seconds of respiratory activity at the respective location that the corresponding strain sensor is attached to. In some embodiments, each physical signal may comprise more than 5 seconds of respiratory activity at the respective location that the corresponding strain sensor is attached to. In some embodiments, each physical signal may comprise 1 to 10 breath cycles. In some embodiments, each physical signal may comprise more than 1 breath cycle. In some embodiments, each physical signal may comprise 5 to 10 breath cycles. In some embodiments, each physical signal may comprise more than 5 breath cycles. The method may further comprise transmitting the plurality of physical signals to the computing device communicatively coupled to the plurality of strain sensors. The method may further comprise the computing device calculating an arithmetic computation of the plurality of physical signals and storing the arithmetic computation of the plurality of physical signals as the baseline respiratory level of the patient. In some embodiments, the arithmetic computation may comprise a sum, average, difference, weighted sum, or a combination thereof of the plurality of physical signals and/or an addition of the plurality of physical signals. In some embodiments, each physical signal and the baseline respiratory level may comprise a respiratory rate, a tidal volume measurement, and a minute ventilation measurement. All future signals will be compared to this baseline respiratory level. The method may further comprise the plurality of strain sensors measuring a second plurality of physical signals. Each physical signal may comprise a continuous waveform of respiratory activity at the respective location. The method may further comprise transmitting the second plurality of physical signals to the computing device and calculating a comparison to the baseline respiratory level. In some embodiments, the comparison may be selected from a group comprising a ratio of each physical signal of the second plurality of physical signals to the baseline respiratory level, and a ratio of a combined signal of the plurality of physical signals to the baseline respiratory level. The method may further comprise triggering an alarm if the ratio of the physical signal to the baseline respiratory level exceeds a threshold. In some embodiments, the threshold may comprise a static value above and a static value below the respiratory rate of the baseline, the tidal volume measurement of the baseline, and the minute ventilation measurement of the baseline. In other embodiments, the threshold may comprise a maximum first derivative of the respiratory rate, the tidal volume measurement, and the minute ventilation measurement such that the plurality of second physical signals cannot exceed a certain speed of change. In some embodiments, the threshold may be determined based on the patient's medical history and recommendations from a medical professional. In some embodiments, the threshold may comprise a plurality of sub-thresholds such that passing a sub-threshold increases a severity level of the triggered alarm. In some embodiments, each physical signal comprises information content at less than 50 Hz. In some embodiments, each physical signal comprises information content at 5 Hz to 50 Hz. In some embodiments, each physical signal comprises information content at less than 20 Hz. In some embodiments, each physical signal comprises information content at 5 Hz to 20 Hz.

In some embodiments, the method may further comprise attaching an additional motion detection device capable of measuring a motion signal to the body of the patient communicatively coupled to the computing device for transmitting the motion signal. In some embodiments, the additional motion detection device may comprise an accelerometer. The present invention may additionally be capable of recomputing the baseline respiratory level. Recomputation may be triggered by a motion signal above a motion threshold, a change in the second plurality of physical signals past a recomputation threshold, user input, or a combination thereof. In some embodiments, recomputation may comprise the plurality of strain sensors measuring a new plurality of physical signals. Each physical signal may comprise 30 to 60 seconds of respiratory activity at the respective location. Recomputation may further comprise transmitting the new plurality of physical signals to the computing device and calculating an arithmetic computation of the new plurality of physical signals. Recomputation may further comprise storing the arithmetic computation of the plurality of physical signals as a new baseline respiratory level of the patient. All future signals will be compared to a combination of this new baseline respiratory level and the original baseline respiratory level. Recomputation may further comprise the plurality of strain sensors measuring the second plurality of physical signals, transmitting the second plurality of physical signals to the computing device, and calculating, for each physical signal of the second plurality of physical signals, an arithmetic combination of signals selected from a group comprising a new ratio of the physical signal compared to the baseline respiratory level, the new ratio multiplied by a last recorded ratio of the physical signal compared to the baseline respiratory level before recomputation of the baseline was initiated, the new ratio multiplied by a constant derived from the new ratio, the new ratio multiplied by a constant derived from the last recorded ratio before recomputation of the baseline was initiated, and a combination thereof. For example, a function could be derived to produce the constant (as the output) based on the previous ratio value's average amplitude over 5 points and change in slope (derivative) over 5 points. In some embodiments, recomputation further comprises the computing device storing data, parameters, and math used in recomputation. The computing device may additionally store whether recomputation was triggered by the motion signal, the second plurality of physical signals, user input, or a combination thereof.

The method may further comprise leaning the patient back in their chair and measuring normal breathing for a period of time. In some embodiments, said period of time is 60 seconds. The doctor may then select a dominant breathing type (chest or abdomen) to determine which sensor to accept data from, and may input a fixed percentage by which the patient's tidal volume may fluctuate before the doctor is alerted of a potentially hazardous tidal volume level, and a fixed percentage by which the patient's minute ventilation may fluctuate before the doctor is alerted of a potentially hazardous minute ventilation level. The RR will always maintain a constant threshold for a potentially hazardous level. In some embodiments, the hazard threshold for both the tidal volume and the minute ventilation may be 30% below the BTV and BMV respectively. The method may further comprise the respiratory measurement sensors measuring the patient's normal breathing, and respiratory data is transmitted based on said breaths in the form of a waveform. From said data, the computing device may generate a baseline measurement from the waveform. The baseline measurement may comprise a RR, calculated by taking the time difference between subsequent peaks in the respiration waveform and dividing said value by 60. The baseline measurement may further comprise a BTV, calculated by subtracting the value of a waveform peak (inhalation) from the value of a subsequent waveform valley (exhalation) over 60 seconds and retrieving the average of all calculated values. The baseline measurement may further comprise a BMV, calculated by multiplying the RR and BTV together for each breath over 60 seconds and retrieving the average of all calculated values. The computing device may then calculate hazard threshold values for the RR, the tidal volume, and the minute ventilation based on the baseline measurements and the doctor-inputted percentage values. The sensors may then continue to transmit respiratory data to the computing device. This respiratory data may comprise a RR, a relative tidal volume, and a relative minute ventilation. Said data may be checked against the hazard threshold values. If a hazard threshold is passed, an alert will appear on the computing device and the doctor will know to intervene at this point. All changes from the baseline level over a period of time may be collected and reported by the computing device in the form of a data file or printed form at the end of said period of time.

What is claimed is:

1. A method for measuring a baseline respiratory level of a patient and comparing future respiratory levels of the patient to the baseline respiratory level to identify a dangerous respiratory status, the method comprising:
a. applying a plurality of strain sensors capable of measuring a plurality of physical signals to a plurality of locations on the patient's body, wherein each physical signal represents an expansion and contraction measurement of the respective location;
b. measuring, by the plurality of strain sensors, the plurality of physical signals, wherein each physical signal comprises 5 to 60 seconds of respiratory activity at the respective location;
c. transmitting the plurality of physical signals to a computing device, wherein the computing device is communicatively coupled to the plurality of strain sensors;
d. calculating, by the computing device, an arithmetic computation of the plurality of physical signals;
e. storing the arithmetic computation of the plurality of physical signals as the baseline respiratory level of the patient;
f. measuring, by the plurality of strain sensors, a second plurality of physical signals, wherein each physical signal comprises a continuous waveform of respiratory activity at the respective location;
g. transmitting the second plurality of physical signals to the computing device;
h. calculating, by the computing device, a comparison to the baseline respiratory level, wherein the comparison is selected from a group comprising a ratio of each physical signal of the second plurality of physical signals to the baseline respiratory level, and a ratio of a combined signal of the plurality of physical signals to the baseline respiratory level; and
i. triggering an alarm if the ratio of the physical signal to the baseline respiratory level exceeds a threshold.

2. The method of claim 1, wherein each physical physical signal comprises information content at less than 50 Hz.

3. The method of claim 1, wherein the plurality of strain sensors comprising a first strain sensor attached to a chest of the patient, and a second strain sensor attached to an abdomen of the patient.

4. The method of claim 1 further comprising attaching an additional motion detection device capable of measuring a motion signal to the body of the patient, wherein the additional motion detection device is communicatively coupled to the computing device for transmitting the motion signal.

5. The method of claim 4, wherein the additional motion detection device comprises an accelerometer.

6. The method of claim 4 further comprising steps for recomputing the baseline based on the patient changing positions, wherein recomputation comprises:
a. measuring, by the plurality of strain sensors, a new plurality of physical signals, wherein each physical signal comprises 30 to 60 seconds of respiratory activity at the respective location;
b. transmitting the new plurality of physical signals to the computing device;
c. calculating, by the computing device, an arithmetic computation of the new plurality of physical signals;
d. storing the arithmetic computation of the plurality of physical signals as a new baseline respiratory level of the patient;
e. measuring, by the plurality of strain sensors, the second plurality of physical signals;
f. transmitting the second plurality of physical signals to the computing device; and
g. calculating, by the computing device, for each physical signal of the second plurality of physical signals, an arithmetic combination of signals selected from a group comprising a new ratio of the physical signal compared to the baseline respiratory level, the new ratio multiplied by a last recorded ratio of the physical signal compared to the baseline respiratory level before recomputation of the baseline was initiated, the new ratio multiplied by a constant derived from the new ratio, the new ratio multiplied by a constant derived from the last recorded ratio before recomputation of the baseline was initiated, and a combination thereof;
wherein recomputation is triggered by a motion signal above a motion threshold, a change in the second plurality of physical signals past a recomputation threshold, user input, or a combination thereof.

7. The method of claim 6 further comprising storing, by the computing device, data, parameters, and math used in recomputation.

8. The method of claim 7 further comprising storing, by the computing device, whether recomputation was triggered by the motion signal, the second plurality of physical signals, user input, or a combination thereof.

9. The method of claim 1, wherein each physical signal and the baseline respiratory level comprises a respiratory rate, a tidal volume measurement, and a minute ventilation measurement.

10. The method of claim 9, wherein the threshold comprises a static value above and a static value below the respiratory rate of the baseline, the tidal volume measurement of the baseline, and the minute ventilation measurement of the baseline.

11. The method of claim 9, wherein the threshold comprises a maximum first derivative of the respiratory rate, the tidal volume measurement, and the minute ventilation measurement such that the plurality of second physical signals cannot exceed a certain speed of change.

12. The method of claims 10-11, wherein the threshold is determined based on the patient's medical history and recommendations from a medical professional.

13. The method of claims 10-11, wherein the threshold comprises a plurality of sub-thresholds such that passing a sub-threshold increases a severity level of the triggered alarm.

14. The method of claim 1, wherein the computing device is selected from a group comprising the plurality of strain sensors, an external computing device, a cloud server, and a combination thereof.

15. The method of claim 1, wherein the arithmetic computation comprises a sum, average, difference, weighted sum, or a combination thereof of the plurality of physical signals.
